(19) United States
(12) Patent Application Publication
(10) Pub. No.: US 2023/0371832 A1
(43) Pub. Date: Nov. 23, 2023

(54) HEALTH METRIC MEASUREMENTS USING HEAD-MOUNTED DEVICE

(71) Applicant: GOOGLE LLC, Mountain View, CA (US)

(72) Inventors: Dongeek Shin, Santa Clara, CA (US); Shwetak N. Patel, Seattle, WA (US)

(21) Appl. No.: 17/664,118

(22) Filed: May 19, 2022

(51) Int. Cl. A61B 5/024; A61B 5/0205; A61B 5/00; G16H 40/63; G16H 50/30

(52) U.S. Cl. CPC A61B 5/02438; A61B 5/0205; A61B 5/02405; A61B 5/6803; A61B 5/721; G16H 40/63; G16H 50/30; A61B 2562/0219

(57) ABSTRACT

The techniques described herein relate to a head-mounted device that includes a frame, a motion sensor coupled to the frame, and a processor in communication with the motion sensor. The processor is configured by instructions to receive motion signals captured by the motion sensor, determine when to extract features from the motion signals, extract the features from the motion signals, generate a signal image from the features extracted from the motion signals, and process the signal image to output one or more health metrics.

### TECHNICAL FIELD

This description relates to a head-mounted device used to measure health metrics.

### BACKGROUND

Health metrics are measures of the body's basic functions. Health metrics may include vital signs such as, for example, heart rate (HR) and respiration rate. Health metrics also may include other clinical metrics such as, for example, heart rate variability, respiration rate variability, bradycardia and tachycardia diagnostics, and atrial fibrillation diagnostics. Measurements of health metrics may be used to assess the clinical situation of a person. The health of a person may be intrusively measured, for example, several times a day, by nurses in a clinical or hospital setting, or at home, or at the site of a medical emergency, etc. Early warning scores (EWS) based on the measured health metrics are generally calculated three times a day in clinical settings, but these may not capture early deterioration.

### SUMMARY

In one general aspect, the techniques described herein relate to a head-mounted device that includes a frame, a motion sensor coupled to the frame, and a processor in communication with the motion sensor. The processor is configured by instructions to receive motion signals captured by the motion sensor, determine when to extract features from the motion signals, extract the features from the motion signals, generate a signal image from the features extracted from the motion signals, and process the signal image to output one or more health metrics.

In another general aspect, the techniques described herein relate to a computer-implemented method, the computer-implemented method including: receiving motion signals captured by a motion sensor on a head-mounted device; determining when to extract features from the motion signals; extracting the features from the motion signals; generating a signal image from the features extracted from the motion signals; and processing the signal image to output one or more health metrics.

In another general aspect, the techniques described herein relate to a computer program product, the computer program product being tangibly embodied on a computer-readable medium and including executable code that, when executed, is configured to cause a processor to: receive motion signals captured by a motion sensor on a head-mounted device; determine when to extract features from the motion signals; extract the features from the motion signals; generate a signal image from the features extracted from the motion signals; and process the signal image to output one or more health metrics.

The details of one or more implementations are set forth in the accompanying drawings and the description below. Other features will be apparent from the description and drawings, and from the claims.

### BRIEF DESCRIPTION OF THE DRAWINGS

FIG. 1 is an example block diagram of a head-mounted device.

FIG. 2 is an example sketch of a head showing the carotid artery.

FIG. 3 is a perspective view of an example head-mounted device of FIG. 1.

FIG. 4 is a perspective view of another example head-mounted device of FIG. 1.

FIG. 5 is an example block diagram of a health metrics application.

FIG. 6 is an example schematic of motion signals illustrating different motion states.

FIG. 7 is a flowchart illustrating example operations of the system of FIG. 1.

FIG. 8 illustrates example computing devices of the computing systems discussed herein.

### DETAILED DESCRIPTION

This document describes systems and techniques for measuring health metrics using a head-mounted device. A head-mounted device configured to measure the health metrics provides an opportunity for monitoring, including remote monitoring, of one or more vital signs and/or clinically relevant health metrics in non-clinical settings. The head-mounted device may allow patients to self-monitor, track, and assess human physiological data, while also providing interfaces (e.g., wireless interfaces) for communicating the health metrics to healthcare providers. Having a head-mounted device for measuring the health metrics decreases any restrictions placed on the patients' mobility and daily activities, and allows monitoring in the patients' natural environments (e.g., at home, at work, or during other activity). Such monitoring of the health metrics with a head-mounted device might detect clinical deterioration at an earlier stage and allow prompt corrective actions.

This document addresses technical problems encountered when using a head-mounted device to measure health metrics. For instance, extra components (e.g., optical components, hardware modules, etc.) on or embedded in the head-mounted device (e.g., on the nose bridge of glasses, in the electronic modules of the device, etc.) may detrimentally interfere with the form, fit, and/or other functions of the head-mounted device. For example, if the head-mounted device is a pair of smart glasses, extra components used to measure and output health metrics may compromise the form, fit, and/or other functions of the smart glasses. Additionally, this document address technical problems encountered for determining when to initiate and/or take the measurements used to calculate and determine the health metrics without having to instruct the wearer to remain still for a period of time or otherwise obtrusively interfere with the wearer's activities. This document provides technical solutions to these and other technical problems.

This document provides technical solutions that use a motion sensor (e.g., a single motion sensor) to capture motion signals related to the movement of the wearer's head as blood pumps through the carotid arteries below the head. The technical solution takes advantage of the observation that a person's head moves periodically as blood pumps through the carotid arteries below the head. As the person's neck functions as an anchor for this pulsatile movement, the motion sensor on the head-mounted device captures the motion signals reflective of this pulsatile movement. The pulsatile movements results in subtle oscillations of the motion sensor on the head-mounted device from the cardiac cycle. The motion sensor captures the features of the swing period information in the motion signals. The technical solution extracts these features (e.g., frequency, time, etc.) from the motion signals and generates a signal image from the features. The signal image is processed to output one or more health metrics. For example, in some implementations, a neural network is used to process the signal image and output the one or more health metrics.

Furthermore, the technical solutions include using the motion signals captured by the motion sensor to determine when to extract the features from the motion signals. The motion signals are processed to determine a motion state of the head-mounted device. In some implementations, a neural network is used to determine the motion state of the head-mounted device. When the motion state of the head-mounted device is in a donned (i.e., the user is wearing the head-mounted device) and static state, the features are extracted from the motion signals and the signal image is generated. When the motion state of the head-mounted device is in a doffed state (i.e., the user is not wearing the head-mounted device) or the motion state of the head-mounted device is in a donned and moving state, the features are not extracted from the motion signals. In this manner, the head-mounted device and its components determine when is an appropriate motion state to extract the features from the motion signals to determine the one or more health metrics. In this manner, the head-mounted device implements a motion-aware gating system that determines when to initiate the process to measure and determine one or more health metrics without obtrusively prompting or otherwise alerting the wearer to take an action to start the process.

As used herein, health metrics refers to and may include vital signs such as, for example, heart rate (HR) (also referred to as pulse rate) and respiration rate. Health metrics also may include other clinical metrics such as, for example, heart rate variability, respiration rate variability, bradycardia and tachycardia diagnostics, and atrial fibrillation diagnostics. Health metrics also may include other vital signs and clinical metrics not specifically listed here.

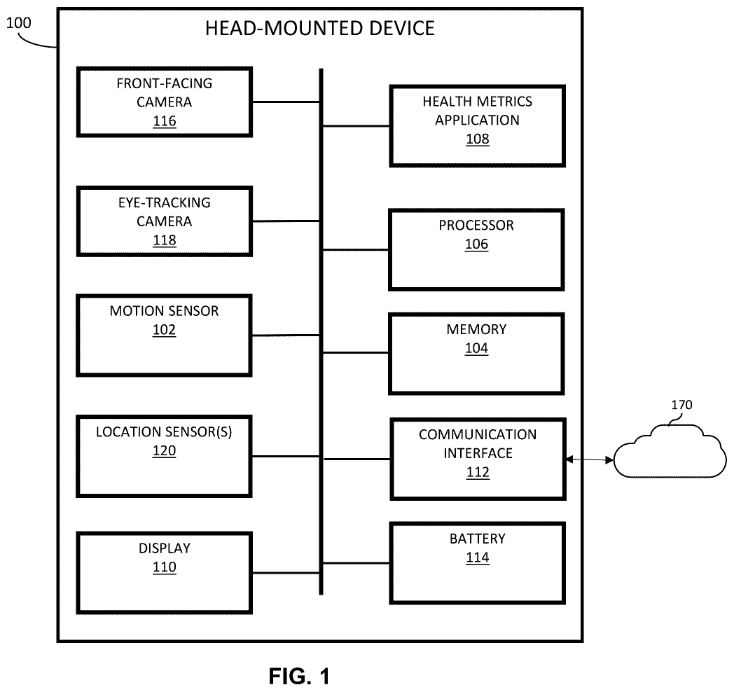

FIG. 1 is an example block diagram of a head-mounted device 100. The head-mounted device 100 may be implemented in different forms including in the form of glasses (also referred to interchangeably as smart glasses), a headset, goggles, or other forms that are worn on the wearer's head. In some implementations, the head-mounted device 100 is in the form of glasses such as ophthalmic eyewear including corrective lenses. In some example, the glasses may be a pair of smart glasses, or augmented reality (AR) or mixed-reality glasses, including display capability and computing/processing capability. The principles to be described herein may be applied to these types of eyewear, and other types of eyewear, both with and without display capability.

The head-mounted device 100 may include a motion sensor 102, a memory 104, a processor 106, a health metrics application 108, a display 110, a communication interface 112, a battery 114, a front-facing camera 116, an eye-tracking camera 118, and a location sensor(s) 120. In some implementations, the head-mounted device 100 may include more or fewer components.

The motion sensor 102 is configured to capture motion (i.e., movement) signals and orientation signals of the head-mounted device 100. The motion signals include movement and oscillations of the wearer's head caused by the pulse of blood through the carotid arteries. That is, every time the heart pumps blood through the body, including through the carotid arteries, the head moves. While the head oscillations caused by blood pumping through the carotid arteries may be imperceptible to the human eye, the motion sensor 102 may capture those head oscillations.

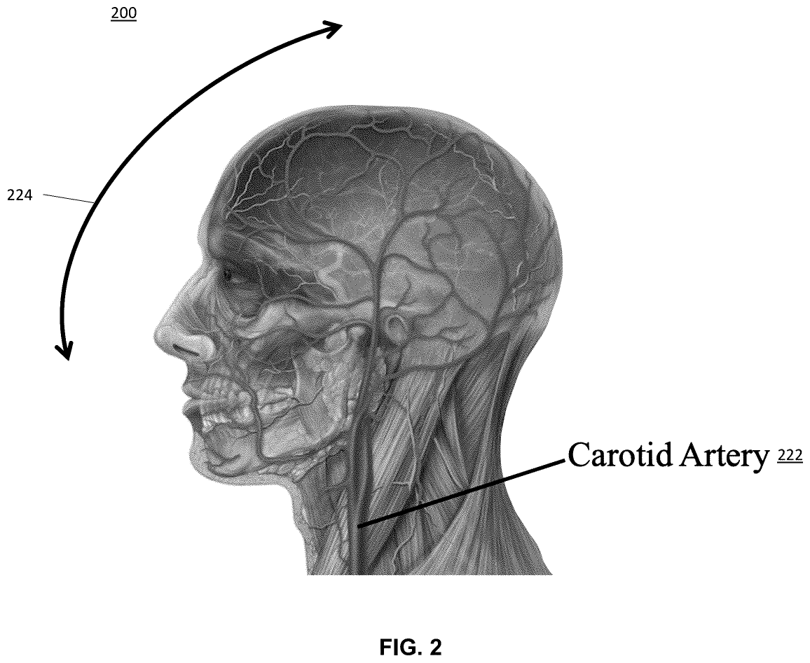

Referring to FIG. 2, an example sketch of a head 200 is illustrated. The head 200 is shown without the skin to illustrate the carotid artery 222 and the flow of blood through the carotid artery 222 throughout the head 200. As mentioned above, each time the heart pumps blood through the body, blood is also pumped through the carotid artery 222, which causes an oscillation or periodic movement of the head 200. The arrow 224 illustrates an example oscillation path or jitter path of the head 200 with each pulse of blood (or heartbeat) through the carotid artery 222. The motion sensor 102 is configured and sensitive enough to capture the movement of the head 200 along the oscillation path indicated by the arrow 224, even though the movements may not be perceptible to the human eye.

Referring back to FIG. 1, the motion sensor 102 may be implemented as an accelerometer, a gyroscope, and/or magnetometer, some of which may be combined to form an inertial measurement unit (IMU). In some implementations, the motion sensor 102 may be implemented as a three-axis motion sensor such as, for example, a three-axis accelerometer or a three-axis gyroscope, where the motion signals captured by the motion sensor 102 describe three translation movements (i.e., x-direction, y-direction, and z-direction) along axes of a world coordinate system.

In some implementations, the motion sensor 102 may be implemented as a six-axis motion sensor such as, for example, an IMU that has six (6) degrees of freedom (6-DOF), which can describe three translation movements (i.e., x-direction, y-direction, and z-direction) along axes of a world coordinate system and three rotation movements (i.e., pitch, yaw, roll) about the axes of the world coordinate system. The IMU motion signals from the IMU may be used to detect and the periodic movements such as the jitters and/or oscillations of the head-mounted device 100 each time that the heart pulses blood through the carotid artery, such as the carotid artery 222 of FIG. 2.

As mentioned above, the motion sensor 102 is configured to detect periodic movements such as the jitters and/or oscillations of the head-mounted device 100 caused by each heart rate or pulse of blood through the carotid arteries. As the neck acts as an anchor for this pulsatile movement, the motion sensor 102 captures or detects the movement from the head-mounted device 100. The jitters and/or oscillations may be referred to as swing period information or swing period data, which may a very low signal-to-noise (SNR) signal in the motion sensor 102 raw data. The swing period information may be used to estimate a wearer's heart rate and other health metrics. The swing period information is recovered from the motion sensor 102 raw data when a static user is wearing the head-mounted device 100 using structure-aware neural denoisers that regresses over the heart rate.

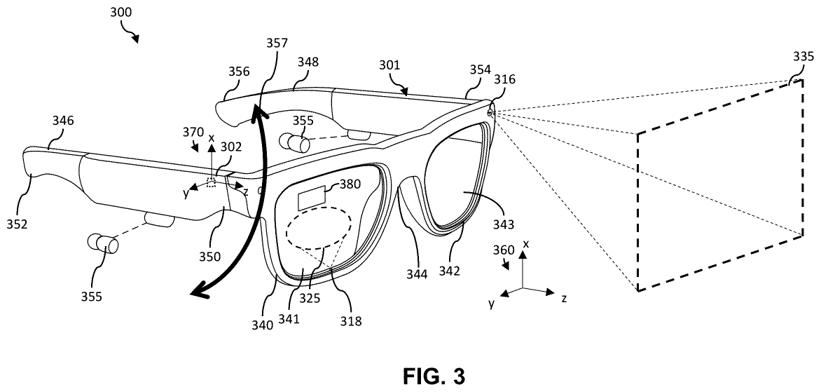

Referring to FIG. 3, an example head-mounted device 300 in the form of glasses is illustrated. The head-mounted device 300 may include the components illustrated in the block diagram of the head-mounted device 100 of FIG. 1. The head-mounted device 300 includes a frame 301 configured to be worn on a head of a user (or wearer). The frame 301 includes a first eye rim 340 holding a first lens 341 and a second eye rim 342 holding a second lens 343. A bridge (or nose bridge) 344 connects the first eye rim 340 and the second eye rim 342. A first temple 346 is connected to the first eye rim 340 and a second temple 348 is connected to the second eye rim 342. The first temple 346 and the second temple 348 are configured so that the frame 301 may rest on the ears of the wearer and the nose bridge 344 is configured so that the frame 301 may rest on the nose of the wearer.

The first temple 346 includes a proximal end 350 and a distal end 352, where the proximal end 350 is the end of the first temple 346 nearer to the first eye rim 340 and the distal end 352 is the end of the first temple 346 away from the first eye rim 340. The distal end 352 also may be referred to as the temple tip. Similarly, the second temple 348 includes a proximal end 354 and a distal end 356, where the proximal end 354 is the end of the second temple 348 nearer to the second eye rim 342 and the distal end 356 is the end of the second temple 348 away from the second eye rim 342. The distal end 356 also may be referred to as the temple tip.

In this example implementation, the head-mounted device 300 includes a motion sensor 302 located on or embedded in the frame 301 at the proximal end 350 of the first temple 346. While this example implementation illustrates the motion sensor 302 on the first temple 346, the motion sensor 302 could also be located on or embedded in the frame 301 at the proximal end 354 of the second temple 348. With the placement of the motion sensor 302 at the proximal end 350 of the first temple 346, the oscillation of the head with each pulse of the blood through the carotid artery is more pronounced compared to the distal end 352 of the first temple 346. The first temple 346 functions as a rigid body that translates the jitter path or oscillation path of the head, as illustrated by the arrow 357, in a more detectable manner with the motion sensor 302 located at the proximal end 350 of the first temple 346. The movement of the head, as indicated by the arrow 357, is more amplified or pronounced at the proximal end 350 of the first temple 346 compared to the distal end 352 of the first temple 346.

The motion sensor 302 may include the features and functions of the motion sensor 102 of FIG. 1. As discussed above with respect to the motion sensor 102, the motion sensor 302 may be implemented as a three-axis motion sensor such as, for example, a three-axis accelerometer or a three-axis gyroscope, where the motion signals captured by the motion sensor 302 describe three translation movements (i.e., x-direction, y-direction, and z-direction) along axes of a world coordinate system 360.

In some implementations, the motion sensor 302 may be implemented as a six-axis motion sensor such as, for example, an IMU that has six (6) degrees of freedom (6-DOF), which can describe three translation movements (i.e., x-direction, y-direction, and z-direction) along axes of a world coordinate system 360 and three rotation movements (i.e., pitch, yaw, roll) about the axes of the world coordinate system 360. Data from the motion sensor 302 can be combined with information regarding the magnetic field of the earth using sensor fusion to determine an orientation of a head-mounted device coordinate system 370 with respect to the world coordinate system 360. The IMU motion signals from the IMU may be used to detect and the periodic movements such as the jitters and/or oscillations of the head-mounted device 100 each time that the heart pulses blood through the carotid artery, such as the carotid artery 222 of FIG. 2.

The head-mounted device 300 may further include human interface subsystems configured to present information to a user. For example, the head-mounted device 300 may include a display, such as the display 110 of FIG. 1, that is configured to display information (e.g., text, graphics, image) in a display area 380 in one or both lenses 341 and 343 as part of a user interface. The display area 380 may be all or part of the lens 341 and may be visually clear or translucent so that when it is not in use the user can view through the display area 380. The head-mounted device 300 may further include one or more speakers (e.g., earbuds 355) configured to play sounds (e.g., voice, music, tones).

The head-mounted device 300 may include sensing devices configured to help determine where a focus of a user is directed. For example, the head-mounted device 300 may include at least one front-facing camera 316 like the front-facing camera 116 of FIG. 1. The front-facing camera 316 can include an image sensor that can detect intensity levels of visible and/or near-infrared light (i.e., MR light). The front-facing camera 316 may be directed towards a front field-of-view (i.e., front FOV 335) or can include optics to route light from the front FOV 335 to the image sensor. For example, the front-facing camera 316 may be positioned in a front surface of the head-mounted device (e.g., on a front surface of the second eye rim 342) and include at least one lens 343 to create an image of a front field-of-view (front FOV 335) on the image sensor. The front FOV 335 may include all (or part) of a field-of-view of the user so that images or video of the world from a point-of-view (POV) of the user may be captured by the front-facing camera 316.

The head-mounted device 300 may further include at least one eye-tracking camera 318 like the eye-tracking camera 118 of FIG. 1. The eye-tracking camera 318 may be directed towards an eye field-of-view (i.e., eye-FOV 325) or can include optics to route light from the eye-FOV 325 to an eye-image sensor. For example, the eye-tracking camera 318 may be directed at an eye of a user and include at least one lens to create an image of the eye-FOV 325 on the eye-image sensor. The eye-FOV 325 may include all (or part) of a field of an eye of the user so that images or video of the eye. The images of the eyes may be analyzed by a processor of the head-mounted device (such as the processor 106 of FIG. 1) to determine where the user is looking. For example, a relative position of the pupil in an image of the eye may correspond to a gaze direction of the user.

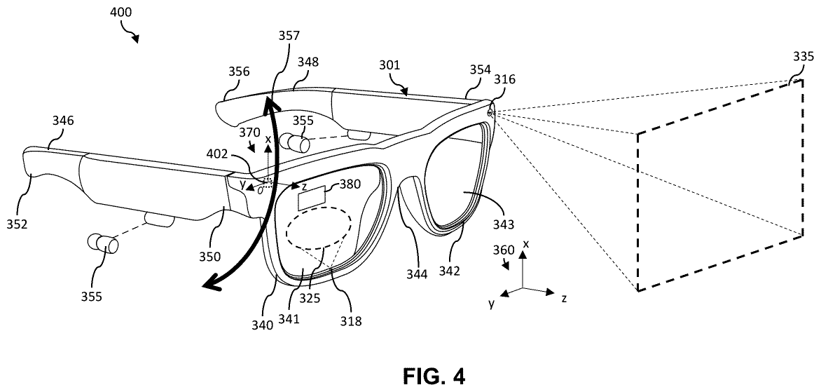

Referring to FIG. 4, another example head-mounted device 400 in the form of glasses is illustrated. The head-mounted device 400 may include the components illustrated in the block diagram of the head-mounted device 100 of FIG. 1 and same components as the head-mounted device 300 of FIG. 3 with the difference being the location of the motion sensor 402 compared to the location of the motion sensor 302 of FIG. 3. The motion sensor 402 includes the same features and functionality as the motion sensor 102 of FIG. 1 and the same features and functionality as the motion sensor 302 of FIG. 3 other than the motion sensor 402 is located on a front surface of the first eye rim 340 instead of on the first temple 346. Although not illustrated, it is understood that the motion sensor 402 alternatively may be placed on a front surface of the second eye rim 342. Placing the motion sensor 402 on the front surface of the first eye rim 340 achieves the same desired result of capturing the motion signals related to head oscillations that occur when the heart pumps blood through the carotid arteries.

Referring back to FIG. 1, the motion sensor 102 captures the motion signals. The memory 104 may store the motion signals for processing by the processor 106. The memory 104 may buffer the motion signals for evaluation and processing by the processor 106 and the health metrics application 108. The memory 104 may include instructions that, when executed by the processor 106, implements the health metrics application 108. The processor 106 and the health metrics application 108 are in communication with the motion sensor 102 and the memory 104. The processor 106 and the health metrics application 108 are configured to receive the motion signals captured by the motion sensor 102.

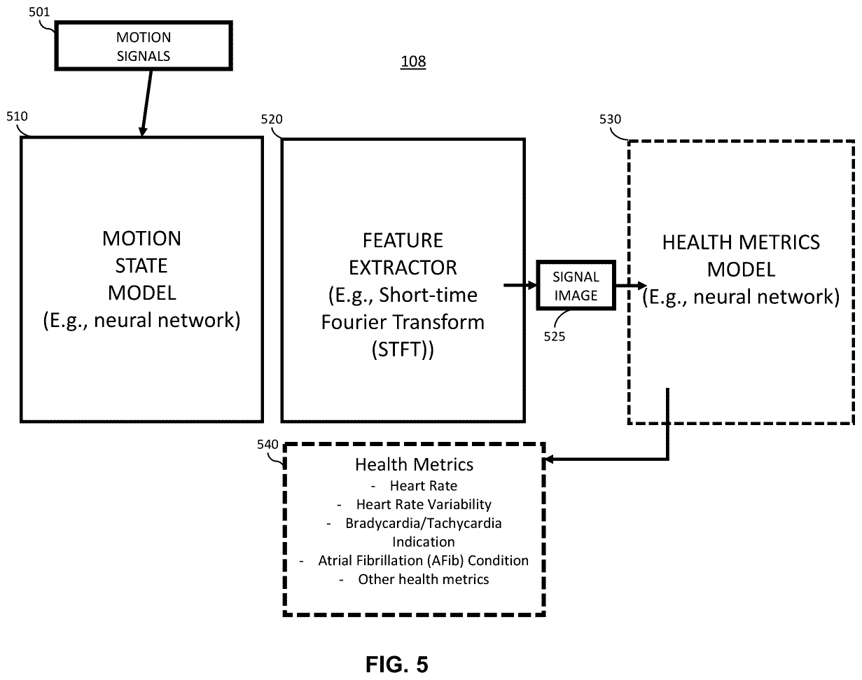

Referring to FIG. 5, an example block diagram of the health metrics application 108 is illustrated. The health metrics application 108 includes a motion state model 510, a feature extractor 520, and a health metrics model 530. In general, the motion signals 501 are received from the motion sensor. The motion state model 510 determines when to extract features from the motion signals 501. When the motion state model 510 determines that the head-mounted device is in a donned and static motion state, as discussed in more detail below, the motion state model 510 passes the motion signals 501 to the feature extractor 520. The feature extractor 520 extracts the features from the motion signals 501 and generates a signal image 525 from the features extracted from the motion signals 501. The health metrics model 530 processes the signal image 525 and outputs one or more health metrics 540, as discussed in more detail below.

In some implementations, the motion state model 510 is configured to determine the motion state of the head-mounted device 100 using the motion signals 501. The motion state model 510 determines whether the head-mounted device 100 is in a doffed state, a donned and moving state, or a donned and static state. In a doffed state, the head-mounted device 100 is not on the user's head. That is, the head-mounted device 100 is not being worn by the user. In a donned and moving state, the head-mounted device 100 is being worn by the user but there is too much movement to extract features from the motion signals and to output accurate and reliable health metrics. In a donned and static state, the head-mounted device 100 is being worn by the user and the head-mounted device 100 is generally static.

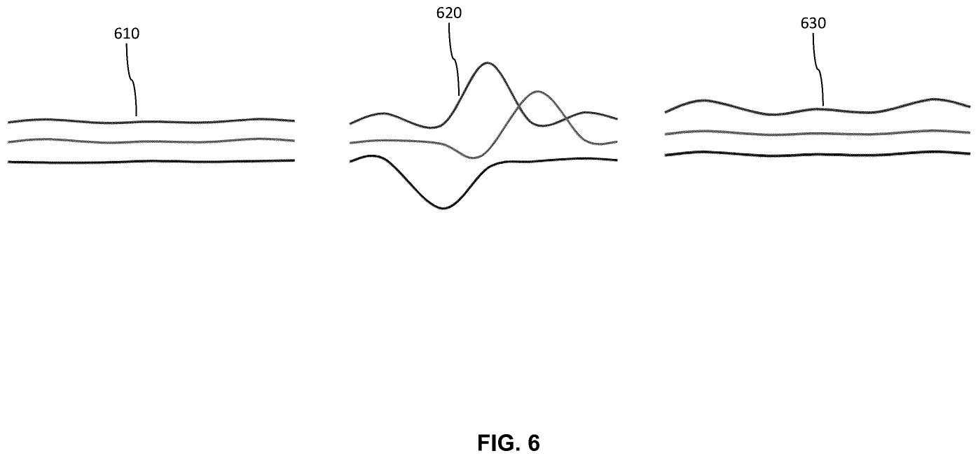

FIG. 6 is an example schematic of motion signals illustrating different motion states. The motion signals 610 illustrate a doffed state. The motion signals 610 are near-perfect constant with the exception of analog-to-digital converter (ADC) noise. The motion signals 620 illustrate a donned and moving state. The motion signals 620 have large swings in amplitude. The motion signals 630 illustrate a donned and static state. The motion signals 630 exhibit residual motion such that features for creating a signal image can be extracted from the motion signals 630.

Referring back to FIG. 5, in some implementations, the motion state model may be implemented as a neural network. The neural network may be a trained in a supervised manner where labeled motion signals are used for training the neural network to predict the motion state using motion signals 501 captured by the motion sensor 102. In this manner, the neural network is trained to predict the motion state of the head-mounted device 100 as either doffed, donned and moving, or donned and static. When the neural network predicts the motion state of the head-mounted device 100 as donned and static, the motion signals 501 are communicated to the feature extractor 520.

In some implementations, when the motion state model 510 is determining the motion state of the head-mounted device 100, the motion signals 501 may be buffered in the memory 104. In this manner, when the motion state model 510 detects the donned and static motion state, the motion signals buffered in the memory 104 may be sent to the feature extractor 520. In some implementations, the motion signals 501 may be buffered for approximately 5 to 15 seconds. In some implementations, the motion signals 501 may be buffered for longer than 15 seconds.

The feature extractor 520 is configured to extract the features from the motion signals 501 and to generate a signal image 525. In some implementations, the feature extractor 520 uses a Short-time Fourier transform (STFT) to extract the features from the motion signals 501. The extracted features may include frequency domain information and time domain information such that a spectrogram may be generated using the STFT. That is, the signal image 525 may be a spectrogram that includes frequency and time signal information. In some implementations, the spectrogram is a multi-axis spectrogram where the number of axis is based on the type of motion sensor. For instance, if the motion sensor is a six-axis IMU, then a six-axis spectrogram may be generated from the motion signals. If the motion sensor is a three-axis accelerometer or a three-axis gyroscope, then a three-axis spectrogram may be generated from the motion signals.

The spectrogram may include a signature horizontal strip concentrated in the appropriate vital signs band, where one vital sign band may be related to information about the heart (e.g., heart rate) and the other vital sign band may be related to respiration. The jitteriness of the vital sign bands indicates vital sign variability. The health metrics for the wearer is determined from the spectrogram by the health metrics model 530.

The signal image 525 is communicated to the health metrics model 530. The health metrics model 530 is configured to process the signal image 525 and to output one or more health metrics 540. In some implementations, the health metrics model 530 is a neural network. The signal image 525, which may be a multi-axis spectrogram, is input into the neural network. The neural network is trained to classify the multi-axis spectrogram and output one or more health metrics 540. The neural network also is trained to perform regression analysis on the spectrogram and output or more health metrics 540. The neural network may include multiple output nodes, where each of the output nodes is for one of the health metrics 540.

In some implementations, the neural network is a multi-target convolutional neural network-long short-term memory (CNN-LSTM) regressor trained in a supervised manner using a clinical IMU dataset with ground truth electrocardiogram (ECG) and/or photoplethysmography (PPG) tagging (or labeling). The neural network may be trained to perform image denoising using neural denoisers to recover the swing period information captured by the motion sensor 102 and represented on the signal image 525. That is, the neural network uses labeled spectrograms from a clinical dataset for training in a supervised manner. The neural network also may be referred to as a convolutional image network, where the neural network is trained to see a lot of such extracted features on the signal image with ground truth vitals information and other health metric information to learn the function that maps the features from the signal image to some compressed health metrics dataset. In this manner, the trained neural network is able to accurately output one more health metrics 540 using both classification and regression.

The health metrics 540 output from the health metrics model 530 may include heart rate, heart rate variability, a bradycardia or a tachycardia condition indication, an atrial fibrillation condition indication, as well as other metrics. Each of the metrics may be represented by a different output node of the health metrics model 530. The heart rate and the heart rate variability are predicted by the health metrics model 530 using regression analysis techniques of the neural network. The bradycardia or tachycardia condition indication and the atrial fibrillation condition indication are predicted by the health metrics model 530 using classification analysis techniques of the neural network.

In some implementations, the heart rate health metric may be output as specific heart rate as measured by the motion sensor capturing motion signals of the movement of the head-mounted device as the heart pulses blood through the carotid artery. In some implementations, the heart rate health metric may be bucketized into a range of a heart rate.

The health metrics 540 may be output to the display 110 on the head-mounted device 100. In some implementations, the health metrics 540 may be output to a device (e.g., a mobile device such as a mobile phone running a corresponding health metrics application) connected to the head-mounted device 100 through the communication interface 112 and the network 170. The health metrics may be displayed on the device connected to or in communication with the head-mounted device 100.

In some implementations, the health metrics model 530 may be implemented on the device connected to the head-mounted device 100. The feature extractor 520 on the head-mounted device 100 may communicate the signal image 525 to the health metrics model running on the device connected to the head-mounted device 100 through the communication interface 112 and the network 170.

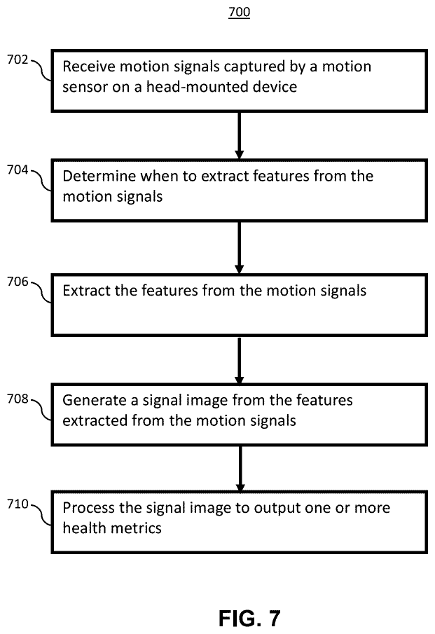

FIG. 7 is an flowchart illustrating an example process 700 of the head-mounted device 100 of FIG. 1. The process 700 may be a computer-implemented method that may be implemented by the head-mounted device 100 of FIG. 1 and its components, including the motion sensor 102, the memory 104, the processor 106, and the health metrics application 108, as well as other components. Instructions and/or executable code for the performance of process 700 may be stored in the memory 104, and the stored instructions may be executed by the processor 106. Process 700 is also illustrative of a computer program product that may be implemented by the head-mounted device 100 and its components, as noted above.

Process 700 includes receiving motion signals captured by a motion sensor on a head-mounted device (702). For example, the processor 106 and/or the health metrics application 108 may receive motion signals captured by the motion sensor 102. More specifically, the motion state model 510 of FIG. 5 may receive the motion signals 501 captured by the motion sensor 102.

Process 700 includes determining when to extract features from the motion signals (704). For example, the processor 106 and/or the health metrics application 108 may determine when to extract features from the motion signals. More specifically, the motion state model 510 of FIG. 5 may determine when to extract features from the motion signals 501. In some implementations, determining when to extract the features from the motion signals 501 includes inputting the motion signals 501 into a neural network and determining a motion state of the head-mounted device 100 using the neural network. In some implementations, the motion state of the head-mounted device 100 include a doffed state, a donned and moving state, and a donned and static state.

Process 700 includes extracting the features from the motion signals (706). For example, the processor 106 and/or the health metrics application 108 may extract the features from the motion signals. More specifically, the feature extractor 520 of FIG. 5 may extract the features from the motion signals 501. In some implementations, the extracting the features from the motion signals 501 includes extracting the features from the motion signals 501 using a Short-time Fourier transform (STFT).

Process 700 includes generating a signal image from the features extracted from the motion signals (708). For example, the processor 106 and/or the health metrics application 108 may generate the signal image from the features extracted from the motion signals. More specifically, the feature extractor 520 of FIG. 5 may generate the signal image 525 from the features extracted from the motion signals 501. In some implementations, generating the signal image includes generating a spectrogram from the features extracted using the STFT.

Process 700 includes processing the signal image to output one or more health metrics (710). For example, the processor 106 and/or the health metrics application 108 may process the signal image to output one or more health metrics. More specifically, the health metrics model 530 of FIG. 5 may process the signal image 525 to output one or more health metrics 540. Processing the signal image 525 to output one or more health metrics may include inputting the signal image 525 into a neural network, determining the one or more health metrics 540 using the neural network, and outputting the one or more health metrics 540 from the neural network for display on a user interface.

In some implementations, processing the signal image to output one or more health metrics (710) may be performed on a device in communication with the head-mounted device 100. The device such as, for example a mobile phone, may include the health metrics model 530 to process the signal image 525 to output the one or more health metrics 540.

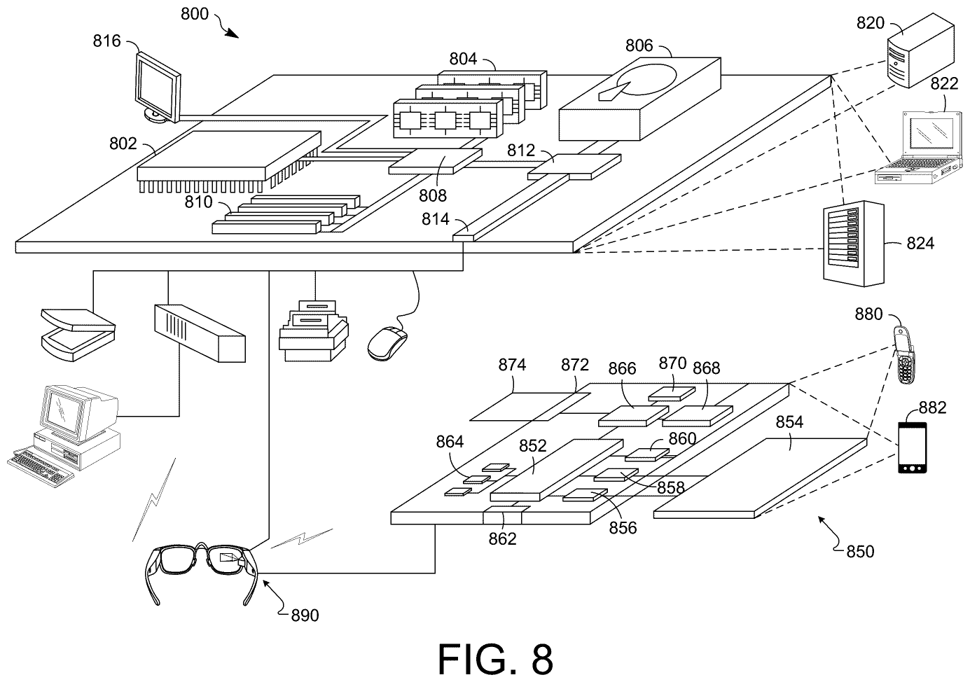

FIG. 8 illustrates an example of a computer device 800 and a mobile computer device 850, which may be used with the techniques described here (e.g., to implement the client computing device and/or the server computing device and/or the provider resources described above). The computing device 800 includes a processor 802, memory 804, a storage device 806, a high-speed interface 808 connecting to memory 804 and high-speed expansion ports 810, and a low-speed interface 812 connecting to low-speed bus 814 and storage device 806. Each of the components 802, 804, 806, 808, 810, and 812, are interconnected using various busses, and may be mounted on a common motherboard or in other manners as appropriate. The processor 802 can process instructions for execution within the computing device 800, including instructions stored in the memory 804 or on the storage device 806 to display graphical information for a GUI on an external input/output device, such as display 816 coupled to high-speed interface 808. In other implementations, multiple processors and/or multiple buses may be used, as appropriate, along with multiple memories and types of memory. Also, multiple computing devices 800 may be connected, with each device providing portions of the necessary operations (e.g., as a server bank, a group of blade servers, or a multi-processor system).

The memory 804 stores information within the computing device 800. In one implementation, the memory 804 is a volatile memory unit or units. In another implementation, the memory 804 is a non-volatile memory unit or units. The memory 804 may also be another form of computer-readable medium, such as a magnetic or optical disk.

The storage device 806 is capable of providing mass storage for the computing device 800. In one implementation, the storage device 806 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 804, the storage device 806, or memory on processor 802.

The high-speed controller 808 manages bandwidth-intensive operations for the computing device 800, while the low-speed controller 812 manages lower bandwidth-intensive operations. Such allocation of functions is example only. In one implementation, the high-speed controller 808 is coupled to memory 804, display 816 (e.g., through a graphics processor or accelerator), and to high-speed expansion ports 810, which may accept various expansion cards (not shown). In the implementation, low-speed controller 812 is coupled to storage device 806 and low-speed expansion port 814. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 800 may be implemented in a number of different forms, as shown in the figure. For example, it may be implemented as a standard server 820, or multiple times in a group of such servers. It may also be implemented as part of a rack server system 824. In addition, it may be implemented in a personal computer such as a laptop computer 822. Alternatively, components from computing device 800 may be combined with other components in a mobile device (not shown), such as device 850. Each of such devices may contain one or more of computing device 800, 850, and an entire system may be made up of multiple computing devices 800, 850 communicating with each other.

Computing device 850 includes a processor 852, memory 864, an input/output device such as a display 854, a communication interface 866, and a transceiver 868, among other components. The device 850 may also be provided with a storage device, such as a microdrive or other device, to provide additional storage. Each of the components 850, 852, 864, 854, 866, and 868, are interconnected using various buses, and several of the components may be mounted on a common motherboard or in other manners as appropriate.

The processor 852 can execute instructions within the computing device 850, including instructions stored in the memory 864. The processor may be implemented as a chipset of chips that include separate and multiple analog and digital processors. The processor may provide, for example, for coordination of the other components of the device 850, such as control of user interfaces, applications run by device 850, and wireless communication by device 850.

Processor 852 may communicate with a user through control interface 858 and display interface 856 coupled to a display 854. The display 854 may be, for example, a TFT LCD (Thin-Film-Transistor Liquid Crystal Display), and LED (Light Emitting Diode) or an OLED (Organic Light Emitting Diode) display, or other appropriate display technology. The display interface 856 may include appropriate circuitry for driving the display 854 to present graphical and other information to a user. The control interface 858 may receive commands from a user and convert them for submission to the processor 852. In addition, an external interface 862 may be provided in communication with processor 852, so as to enable near area communication of device 850 with other devices. External interface 862 may provide, for example, for wired communication in some implementations, or for wireless communication in other implementations, and multiple interfaces may also be used.

The memory 864 stores information within the computing device 850. The memory 864 can be implemented as one or more of a computer-readable medium or media, a volatile memory unit or units, or a non-volatile memory unit or units. Expansion memory 874 may also be provided and connected to device 850 through expansion interface 872, which may include, for example, a SIMM (Single In-Line Memory Module) card interface. Such expansion memory 874 may provide extra storage space for device 850, or may also store applications or other information for device 850. Specifically, expansion memory 874 may include instructions to carry out or supplement the processes described above, and may include secure information also. Thus, for example, expansion memory 874 may be provided as a security module for device 850, and may be programmed with instructions that permit secure use of device 850. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 864, expansion memory 874, or memory on processor 852, that may be received, for example, over transceiver 868 or external interface 862.

Device 850 may communicate wirelessly through communication interface 866, which may include digital signal processing circuitry where necessary. Communication interface 866 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 868. In addition, short-range communication may occur, such as using a Bluetooth, Wi-Fi, or other such transceiver (not shown). In addition, GPS (Global Positioning System) receiver module 870 may provide additional navigation- and location-related wireless data to device 850, which may be used as appropriate by applications running on device 850.

Device 850 may also communicate audibly using audio codec 860, which may receive spoken information from a user and convert it to usable digital information. Audio codec 860 may likewise generate audible sound for a user, such as through a speaker, e.g., in a handset of device 850. Such sound may include sound from voice telephone calls, may include recorded sound (e.g., voice messages, music files, etc.) and may also include sound generated by applications operating on device 850.

The computing device 850 may be implemented in a number of different forms, as shown in the figure. For example, it may be implemented as a cellular telephone 880. It may also be implemented as part of a smartphone 882, personal digital assistant, or other similar mobile device.

In the following, some examples are described.
Example 1: A head-mounted device that includes a frame, a motion sensor coupled to the frame, and a processor in communication with the motion sensor. The processor is configured by instructions to receive motion signals captured by the motion sensor, determine when to extract features from the motion signals, extract the features from the motion signals, generate a signal image from the features extracted from the motion signals, and process the signal image to output one or more health metrics.
Example 2: The head-mounted device of example 1, wherein: determining when to extract the features from the motion signals includes: inputting the motion signals into a neural network, and determining a motion state of the head-mounted device using the neural network; and extracting the features from the motion signals is initiated based on the motion state of the head-mounted device determined by the neural network.
Example 3: The head-mounted device of example 1 or 2, wherein extracting the features from the motion signals includes extracting the features from the motion signals using a Short-time Fourier transform (STFT).
Example 4: The head-mounted device of example 3, wherein generating the signal image includes generating a spectrogram from the features extracted using the STFT.
Example 5: The head-mounted device of any of the preceding examples, wherein processing the signal image to output the one or more health metrics includes: inputting the signal image into a neural network; determining the one or more health metrics using the neural network; and outputting the one or more health metrics from the neural network for display on a user interface.
Example 6: The head-mounted device of any of the preceding examples, wherein the one or more health metrics include a heart rate.
Example 7: The head-mounted device of any of the preceding examples, wherein the one or more health metrics includes a heart rate variability.
Example 8: The head-mounted device of any of the preceding examples, wherein the one or more health metrics includes an atrial fibrillation condition indication.
Example 9: The head-mounted device of any of the preceding examples, wherein: the frame includes: a first eye rim, a second eye rim, a bridge connecting the first eye rim and the second eye rim, a first temple connected to the first eye rim, and a second temple connected to the second eye rim; and the motion sensor is embedded in the first temple near the first eye rim.
Example 10: The head-mounted device of any of examples 1 through 8, wherein: the frame includes: a first eye rim, a second eye rim, and a bridge connecting the first eye rim and the second eye rim; and the motion sensor is embedded in the first eye rim.
Example 11: The head-mounted device of any of the preceding examples, wherein the motion sensor includes a six-axis inertial measurement unit (IMU).
Example 12: The head-mounted device of example 11, wherein the six-axis IMU includes an accelerometer and a gyroscope.
Example 13: The head-mounted device of any of examples 1 through 10, wherein the motion sensor includes a three-axis motion sensor.
Example 14: The head-mounted device of example 13, wherein the three-axis motion sensor includes an accelerometer.
Example 15: A computer-implemented method, the computer-implemented method including: receiving motion signals captured by a motion sensor on a head-mounted device; determining when to extract features from the motion signals; extracting the features from the motion signals; generating a signal image from the features extracted from the motion signals; and processing the signal image to output one or more health metrics.
Example 16: The computer-implemented method of example 15, wherein: determining when to extract the features from the motion signals includes: inputting the motion signals into a neural network, and determining a motion state of the head-mounted device using the neural network; and extracting the features from the motion signals is initiated based on the motion state of the head-mounted device determined by the neural network.
Example 17: The computer-implemented method of example 15 or 16, wherein processing the signal image to output the one or more health metrics includes: inputting the signal image into a neural network; determining the one or more health metrics using the neural network; and outputting the one or more health metrics from the neural network for display on a user interface.
Example 18: A computer program product, the computer program product being tangibly embodied on a computer-readable medium and including executable code that, when executed, is configured to cause a processor to: receive motion signals captured by a motion sensor on a head-mounted device; determine when to extract features from the motion signals; extract the features from the motion signals; generate a signal image from the features extracted from the motion signals; and process the signal image to output one or more health metrics.
Example 19: The computer program product of example 18, wherein: determining when to extract the features from the motion signals includes: inputting the motion signals into a neural network, and determining a motion state of the head-mounted device using the neural network; and extracting the features from the motion signals is initiated based on the motion state of the head-mounted device determined by the neural network.
Example 20: The computer program product of example 18 or 19, wherein processing the signal image to output the one or more health metrics includes: inputting the signal image into a neural network; determining the one or more health metrics using the neural network; and outputting the one or more health metrics from the neural network for display on a user interface.
Example 21: A computer-implemented method, the computer-implemented method including: receiving motion signals captured by a motion sensor on a head-mounted device; means for determining when to extract features from the motion signals; means for extracting the features from the motion signals; means for generating a signal image from the features extracted from the motion signals; and means for processing the signal image to output one or more health metrics.

Specific structural and functional details disclosed herein are merely representative for purposes of describing example embodiments. Example embodiments, however, may be embodied in many alternate forms and should not be construed as limited to only the embodiments set forth herein.

The terminology used herein is for the purpose of describing particular embodiments only and is not intended to be limiting of the embodiments. As used herein, the singular forms “a,” “an,” and “the” are intended to include the plural forms as well, unless the context clearly indicates otherwise. It will be further understood that the terms “comprises,” “comprising,” “includes,” and/or “including,” when used in this specification, specify the presence of the stated features, steps, operations, elements, and/or components, but do not preclude the presence or addition of one or more other features, steps, operations, elements, components, and/or groups thereof.

It will be understood that when an element is referred to as being “coupled,” “connected,” or “responsive” to, or “on,” another element, it can be directly coupled, connected, or responsive to, or on, the other element, or intervening elements may also be present. In contrast, when an element is referred to as being “directly coupled,” “directly connected,” or “directly responsive” to, or “directly on,” another element, there are no intervening elements present. As used herein the term “and/or” includes any and all combinations of one or more of the associated listed items.

Spatially relative terms, such as “beneath,” “below,” “lower,” “above,” “upper,” and the like, may be used herein for ease of description to describe one element or feature in relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 70 degrees or at other orientations) and the spatially relative descriptors used herein may be interpreted accordingly.

Example embodiments of the concepts are described herein with reference to cross-sectional illustrations that are schematic illustrations of idealized embodiments (and intermediate structures) of example embodiments. As such, variations from the shapes of the illustrations as a result, for example, of manufacturing techniques and/or tolerances, are to be expected. Thus, example embodiments of the described concepts should not be construed as limited to the particular shapes of regions illustrated herein but are to include deviations in shapes that result, for example, from manufacturing. Accordingly, the regions illustrated in the figures are schematic in nature and their shapes are not intended to illustrate the actual shape of a region of a device and are not intended to limit the scope of example embodiments.

It will be understood that although the terms “first,” “second,” etc. may be used herein to describe various elements, these elements should not be limited by these terms. These terms are only used to distinguish one element from another. Thus, a “first” element could be termed a “second” element without departing from the teachings of the present embodiments.

Unless otherwise defined, the terms (including technical and scientific terms) used herein have the same meaning as commonly understood by one of ordinary skill in the art to which these concepts belong. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

Implementations of the various techniques described herein may be implemented in digital electronic circuitry, or in computer hardware, firmware, software, or in combinations of them. Implementations may be implemented as a computer program product, i.e., a computer program tangibly embodied in an information carrier, e.g., in a machine-readable storage device, for execution by, or to control the operation of, data processing apparatus, e.g., a programmable processor, a computer, or multiple computers. A computer program, such as the computer program(s) described above, can be written in any form of programming language, including compiled or interpreted languages, and can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

Method steps may be performed by one or more programmable processors executing a computer program to perform functions by operating on input data and generating output. Method steps also may be performed by, and an apparatus may be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. Elements of a computer may include at least one processor for executing instructions and one or more memory devices for storing instructions and data. Generally, a computer also may include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory may be supplemented by, or incorporated in, special purpose logic circuitry.

To provide for interaction with a user, implementations may be implemented on a computer having a display device, e.g., a cathode ray tube (CRT) or liquid crystal display (LCD) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

Implementations may be implemented in a computing system that includes a back-end component, e.g., as a data server, or that includes a middleware component, e.g., an application server, or that includes a front-end component, e.g., a client computer having a graphical user interface or a Web browser through which a user can interact with an implementation, or any combination of such back-end, middleware, or front-end components. Components may be interconnected by any form or medium of digital data communication, e.g., a communication network. Examples of communication networks include a local area network (LAN) and a wide area network (WAN), e.g., the Internet.

While certain features of the described implementations have been illustrated as described herein, many modifications, substitutions, changes, and equivalents will now occur to those skilled in the art. It is, therefore, to be understood that the appended claims are intended to cover such modifications and changes as fall within the scope of the implementations. It should be understood that they have been presented by way of example only, not limitation, and various changes in form and details may be made. Any portion of the apparatus and/or methods described herein may be combined in any combination, except mutually exclusive combinations. The implementations described herein can include various combinations and/or sub-combinations of the functions, components, and/or features of the different implementations described.

What is claimed is:

1. A head-mounted device comprising:
a frame;
a motion sensor coupled to the frame; and
a processor in communication with the motion sensor, the processor configured by instructions to:
receive motion signals captured by the motion sensor,
determine when to extract features from the motion signals,
extract the features from the motion signals,
generate a signal image from the features extracted from the motion signals, and
process the signal image to output one or more health metrics.

2. The head-mounted device of claim 1, wherein:
determining when to extract the features from the motion signals comprises:
inputting the motion signals into a neural network, and
determining a motion state of the head-mounted device using the neural network; and
extracting the features from the motion signals is initiated based on the motion state of the head-mounted device determined by the neural network.

3. The head-mounted device of claim 1, wherein extracting the features from the motion signals includes extracting the features from the motion signals using a Short-time Fourier transform (STFT).

4. The head-mounted device of claim 3, wherein generating the signal image includes generating a spectrogram from the features extracted using the STFT.

5. The head-mounted device of claim 1, wherein processing the signal image to output the one or more health metrics comprises:
inputting the signal image into a neural network;
determining the one or more health metrics using the neural network; and
outputting the one or more health metrics from the neural network for display on a user interface.

6. The head-mounted device of claim 1, wherein the one or more health metrics include a heart rate.

7. The head-mounted device of claim 1, wherein the one or more health metrics includes a heart rate variability.

8. The head-mounted device of claim 1, wherein the one or more health metrics includes an atrial fibrillation condition indication.

9. The head-mounted device of claim 1, wherein:
the frame comprises:
a first eye rim,
a second eye rim,
a bridge connecting the first eye rim and the second eye rim,
a first temple connected to the first eye rim, and
a second temple connected to the second eye rim; and
the motion sensor is embedded in the first temple near the first eye rim.

10. The head-mounted device of claim 1, wherein:
the frame comprises:
a first eye rim,
a second eye rim, and
a bridge connecting the first eye rim and the second eye rim; and
the motion sensor is embedded in the first eye rim.

11. The head-mounted device of claim 1, wherein the motion sensor includes a six-axis inertial measurement unit (IMU).

12. The head-mounted device of claim 11, wherein the six-axis IMU includes an accelerometer and a gyroscope.

13. The head-mounted device of claim 1, wherein the motion sensor includes a three-axis motion sensor.

14. The head-mounted device of claim 13, wherein the three-axis motion sensor includes an accelerometer.

15. A computer-implemented method, the computer-implemented method comprising:
receiving motion signals captured by a motion sensor on a head-mounted device;
determining when to extract features from the motion signals;
extracting the features from the motion signals;
generating a signal image from the features extracted from the motion signals; and
processing the signal image to output one or more health metrics.

16. The computer-implemented method of claim 15, wherein:
determining when to extract the features from the motion signals comprises:
inputting the motion signals into a neural network, and
determining a motion state of the head-mounted device using the neural network; and
extracting the features from the motion signals is initiated based on the motion state of the head-mounted device determined by the neural network.

17. The computer-implemented method of claim 15, wherein processing the signal image to output the one or more health metrics comprises:
inputting the signal image into a neural network;
determining the one or more health metrics using the neural network; and
outputting the one or more health metrics from the neural network for display on a user interface.

18. A computer program product, the computer program product being tangibly embodied on a computer-readable medium and including executable code that, when executed, is configured to cause a processor to:
receive motion signals captured by a motion sensor on a head-mounted device;
determine when to extract features from the motion signals;
extract the features from the motion signals;
generate a signal image from the features extracted from the motion signals; and
process the signal image to output one or more health metrics.

19. The computer program product of claim 18, wherein:
determining when to extract the features from the motion signals comprises:
inputting the motion signals into a neural network, and
determining a motion state of the head-mounted device using the neural network; and
extracting the features from the motion signals is initiated based on the motion state of the head-mounted device determined by the neural network.

20. The computer program product of claim 18, wherein processing the signal image to output the one or more health metrics comprises:
inputting the signal image into a neural network;
determining the one or more health metrics using the neural network; and
outputting the one or more health metrics from the neural network for display on a user interface.
